(19) United States
(12) Patent Application Publication
(10) Pub. No.: US 2002/0085978 A1
(43) Pub. Date: Jul. 4, 2002

(54) Degradation-resistant glucocorticosteroid formulations

(76) Inventors: Mina Buenafe, Miami Beach, FL (US); Michael Brucato, Miami Shores, FL (US); Kai Zhang, Miami, FL (US)

Correspondence Address: BAKER NORTON PHARMACEUTICALS, INC., 4400 Biscayne Boulevard, Miami, FL, 33137, US

(21) Appl. No.: 09/768,915

(22) Filed: Jan. 24, 2001

Related U.S. Application Data
(60) Provisional application No. 60/247,361, filed on Nov. 10, 2000.

(51) Int. Cl. A61L 9/04; A61K 31/573; A61K 31/375

(52) U.S. Cl. 424/45; 514/171; 514/474

(57) ABSTRACT

The present invention thus provides chemically and physically stable formulations of glucocorticosteroids (such as, for example, budesonide) obtained by formulating the glucocorticosteroid with a cosolvent (such as ethanol), a propellant (such as HFA-134a, HFA-227ea, or both), and a radical quencher (such as ascorbyl palmitate), where the glucocorticosteroid remains chemically and physically stable under standard conditions

### BACKGROUND OF THE INVENTION

1. Field of the Invention

The invention relates to novel pharmaceutical formulations containing a glucocorticosteroid.

2. Summary of the Related Art

Glucocorticosteroids, and pharmaceutical formulations thereof, are useful medicaments in the treatment of various ailments including bronchial disorders and inflammatory bowel disorders. However, current formulations of glucocorticosteroids are chemically unstable, resulting in costly and inconvenient storage limitations. The degradation (e.g., oxidation and/or hydrolysis) of glucocorticosteroids, when in contact with an organic or inorganic solvent, results in chemical instability.

U.S. Pat. No. 5,914,122 (Otterbeck et al.) discloses a solution, with a pH of at most 6.0, of a glucocorticosteroids (budesonide) dissolved in a solvent (alcohol, water, or a mixture thereof), which may also include a preservative such as ethylenediamine-tetraacetic acid, cyclodextrins, or a mixture thereof. The preferred concentration of budesonide in the formulations of Otterbeck et al. is between 0.01% and 0.1% by weight (at col. 4, lines 31-33). Otterbeck et al. does not disclose a solution of a glucocorticosteroid containing any ingredient other than the preservatives ethylenediamine-tetraacetic acid, cyclodextrins, or a mixture thereof that inhibits degradation of the glucocorticosteroid.

Several investigators have sought to address the issue of the physical and chemical instability of the crystalline form of glucocorticosteroid powder formulations. For example, U.S. Pat. No. 5,874,063 (Briggner et a.) discloses a suspension type aerosol formulation in the form of particles comprising a medicament and an excipient, such as a carbohydrate, an amino acid, or an antioxidant. The particles of Briggner et al. are further treated to enhance their stability with a solvent, such as water or an organic solvent (e.g., an alcohol), where the excess solvent is removed from the particles. Briggner et al. is primarily concerned with the stability of the powder form. This patent does not address the degradation of a glucocorticosteroid in a solution or a suspension formulation for administration using a metered dose inhaler (“MDI”). More important, Briggner et al does not teach or suggest formulations, which are stabilized by the addition of stabilizing moieties.

Similarly, U.S. Pat. No. 5,709,884 (Trofast et al) teaches a process for conditioning of medicament and excipients in a formulation suitable for inhalation. Trofast et al. is primarily concerned with the physical stability of the crystalline form of raw material components to be later formulated in powder form. Hence, Trofast et al. does not teach or suggest formulations, which are chemically stabilized by the addition of stabilizing moieties. This patent does not address the degradation of a glucocorticosteroid in a solution or a suspension formulation for administration using a metered dose inhaler.

Therefore, there remains a need for more stable glucocorticosteroid formulations that resist degradation and display improved chemical and physical stability profiles under standard conditions.

### SUMMARY OF THE INVENTION

The present invention provides novel pharmaceutical formulations of glucocorticosteroids that resist degradation and display improved chemical and physical stability profiles under standard conditions.

Accordingly, in a first aspect, the invention provides a pharmaceutical composition comprising a glucocorticosteroid, a propellant, a cosolvent, and a radical quencher.

In a second aspect, the invention provides a pressurized metered dose inhaler comprising a container equipped with a metering valve and containing a pressurized aerosol formulation comprising a glucocorticosteroid, a propellant, a cosolvent, and a radical quencher.

A third aspect of the invention provides a method for the treatment of a bronchial disorder or an inflammatory bowel disorder in a mammal by administering a pharmaceutical formulation comprising a glucocorticosteroid, a propellant, a cosolvent, and a radical quencher.

In certain preferred embodiments of any of the three aspects of the present invention, the radical quencher is, without limitation, ascorbyl palmitate or Vitamin E.

The glucocorticosteroid included in the various embodiments of the invention may be budesonide, testosterone, progesterone, estrogen, flunisolide, triamcinolone, beclomethasone, betamethasone, dexamethasone, fluticasone, methylprednisolone, prednisone, hydrocortisone, ciclesonide, mometasone, desonide, or rofleponide. Preferably, the glucocorticosteroid is budesonide.

The propellant included in the various embodiments are 1,1,1,2-tetrafluoroethane (“HFA-134a”), 1,1,1,2,3,3,3-heptafluoro-n-propane (“HFA-227ea”) or a mixture thereof.

In various embodiments of the above aspects of the invention, the cosolvent is polyol. In certain embodiments, the polyol is a C₂-C₆ alcohol. Particularly, the preferred polyols are ethanol, isopropanol, or propylene glycol.

### BRIEF DESCRIPTION OF THE DRAWINGS

FIG. 1 is an HPLC chromatogram of a control formulation (Formulation A) containing budesonide, ethanol, and HFA-134a, but lacking a radical quencher according to the invention, which was stored at 40° C. for 28 days.

FIG. 2 is a sample HPLC chromatogram of a representative formulation of the invention containing budesonide, ethanol, HFA-134a, and ascorbyl palmitate as a radical quencher, which was stored at 40° C. for 28 days.

FIG. 3 is a graph displaying the total percent degradation of Formulations A and B in coated and uncoated canisters (AU=Formulation A/uncoated, BU=Formulation B/uncoated, AC=Formulation A/coated, and BC=Formulation B/coated) over a period of 0-180 days at 40° C. of oven treatment.

### DETAILED DESCRIPTION OF THE PREFERRED EMBODIMENTS

The inventors have made the unexpected discovery that the addition of a radical quencher results in a formulation that resists degradation and displays improved chemical and physical stability profiles under standard conditions.

The present invention thus provides chemically and physically stable formulations of glucocorticosteroids (such as, for example, budesonide) obtained by formulating the glucocorticosteroid with a cosolvent (such as ethanol), a propellant (such as HFA-134a, HFA-227ea, or both), and a radical quencher (such as ascorbyl palmitate), where the glucocorticosteroid remains chemically and physically stable under standard conditions. The patent and scientific literature referred to herein established the knowledge that is available to those with skill in the art. The issued U.S. patents, allowed patent application, and articles cited herein are hereby incorporated in their entirety.

Accordingly, in a first aspect, the invention provides a pharmaceutical composition which remains chemically and physically stable under standard conditions comprising a glucocorticosteroid, a propellant, a cosolvent, and a radical quencher.

In a second aspect, the invention provides a pressurized metered dose inhaler comprising a container equipped with a metering valve and containing a pressurized aerosol formulation comprising a glucocorticosteroid, a propellant, a cosolvent, and a radical quencher.

A third aspect of the invention provides a method for the treatment of a bronchial disorder or an inflammatory bowel disorder in a mammal by administering a pharmaceutical formulation comprising a glucocorticosteroid, a propellant, a cosolvent, and a radical quencher.

Technical and scientific terms used herein have the meaning commonly understood by one of skill in the art to which the present invention pertains, unless defined otherwise. Reference is made herein to various methodologies and materials known to those of skill in the art. Standard reference works setting forth the general principles of pharmacology include Goodman and Gilman's The Pharmacological Basis of Therapeutics, 9^{th} Ed., McGraw Hill Companies Inc., New York (1996). Standard reference works setting forth the general principles of pharmaceutical formulations include Remington's Pharmaceutical Sciences, 18^{th} Ed., Gennaro, Mack Publishing Co., Easton, Pa. (1990) and Remington: The Science and Practice of Pharmacy, Lippincott, Williams & Wilkins (1995). Another standard reference works setting forth inhalation technology include the “Pharmaceutical Inhalation Aerosol Technology”, edited by Anthony J. Hickey, Marcel Dekker, Inc., New York, N.Y. (1992).

Any suitable materials and/or methods known to those of skill can be utilized in carrying out the present invention. However, preferred materials and methods are described. Materials, reagents and the like to which reference is made in the following description and examples are obtainable from commercial sources, unless otherwise noted.

The methods of the present invention are intended for use with any “mammal” which may experience the benefits of the methods of the invention. Foremost among such mammals are humans, although the invention is not intended to be so limited. . A “mammal” also includes animals, and is applicable to veterinary uses.

A “functional equivalent” of a biochemical moiety, is a molecule that possesses a biological activity (either functional or structural) that is substantially similar to a biological activity for the moiety of which it is said to be a functional equivalent. The term “functional equivalent” also includes the functional derivatives of any given glucocorticosteroid and modifications for the performance of a specific function. Accordingly, for example, a functional equivalent may contain additional chemical moieties not normally a part of the molecule to which it is a functional equivalent. Such moieties can improve the molecule's solubility, absorption, biological half-life, pharmacokinetic absorption and adsorption, and the like. The moieties can alternatively decrease the toxicity of the molecule, eliminate or attenuate any undesirable side effect of the molecule, and the like. Moieties capable of mediating such effects are disclosed in Remington's Pharmaceutical Sciences (see supra). Procedures for coupling such moieties to a molecule are well known in the art.

The term “functional derivative” is intended to include chemical derivatives of a molecule.

“Standard conditions” as used herein denotes 25° C. and 60% relative humidity. It has been widely accepted that for evaluation purposes incubation for two months at 30° C. and 60% relative humidity are intermediate conditions equivalent to exposure for three months at room temperature (30° C./60% relative humidity). Similarly, one month in accelerated conditions at 40° C. and 75% relative humidity represents an equivalent exposure for four months at room temperature (25° C./60% relative humidity).

A “container”, is a vessel capable of withstanding the vapor pressure of the propellant used such as a plastic-coated glass bottle or aluminum can.

A “pressurized metered dose inhaler” as used herein is designed to deliver a fixed unit dosage of medicament per actuation or “puff”, for example in the range of 10-5000 micrograms of medicament per puff.

A “pressurized aerosol formulation”, is a composition or formulation that is adjusted within a container to have a specific vapor pressure which is measured by the units of psi at a certain temperature.

A “metering valve”, is designed to deliver a metered amount of the formulation per actuation and incorporate a gasket to prevent leakage of propellant through the valve. Suitable valves are commercially available from manufacturers well known in the aerosol industry.

In accordance with the invention, by “glucocorticosteroid” is meant a steroid that is either produced by the adrenal-cortex, or is chemically synthesized such that it functionally mimics a steroid produced by the adrenal cortex. A glucocorticosteroid of the invention includes, without limitation, budesonide, testosterone, progesterone, estrogen, flunisolide, triamcinolone, beclomethasone (e.g., as the mono or the dipropionate ester), betamethasone; dexamethasone, fluticasone (e.g., as the propionate ester), methylprednisolone, prednisone, hydrocortisone, ciclesonide, mometasone, desonide, or rofleponide (i.e., (22R)-6α,9α-difluoro-11β,21-dihydroxy-16α,17α-propylmethylenedioxy-4-pregnen-3,20-dione), and functional equivalents and functional derivatives thereof. Preferred compositions of the invention comprise budesonide.

The amount of glucocorticosteroid utilized in the present formulations is usually from about 0.01 to about 1% by weight, preferably from about 0.05 to about 0.5% by weight, and most preferably about 0.3% by weight, based on the total weight of the aerosol formulation. All weight percentages described herein are based on the total weight of the formulation unless stated otherwise.

A “bronchial disorder” is meant by an inflammation or obstruction of the bronchi, bronchioles, and lung.

An “inflammatory bowel disorder” is meant by an inflammation of the colon, the proximal portion of the intestine distal to the stomach, which also includes the duodenium, jejunum, and ileum.

The present invention also includes formulations containing non-steroidal bronchodilators. By “bronchodilator” is meant a medicament or drug that relaxes bronchial muscle resulting in expansion of the bronchial air passages. Included as non-glucocorticosteroid bronchodilators are, without limitation, β₂-adrenergic agonists, such as albuterol, bambuterol, terbutaline, fenoterol, clenbuterol, procaterol, bitolterol, and brodxaterol; anticholinergic bronchodilators, such as ipratropium bromide and oxytropium bromide. Other non-glucocorticosteroid bronchodilators include formoterol, salmeterol, and TA 2005 (i.e., 8-hydroxy-5-(1-hyroxy-2-2((2-(4-methoxyphenyl)-1-methylethyl)amino)ethyl)-2(1H)-quinolinone) (e.g., as the monohydrochloride), as well as anti-histamines (e.g., terfenadine). One of skill in the art of pulmonary pharmaceuticals will appreciate that the bronchodilators described herein include also functional equivalents and/or derivatives thereof.

In accordance with the invention, a “radical quencher” is meant by a substance capable of inhibiting radical formation, either by reducing radicals already formed or by preventing radical formation. Numerous radical quenchers are known and include, without limitation, ascorbic acid, ascorbyl palmitate, sodium bisulfite, butylated hydroxytoluene (BHT), bytylated hydroxyanisole (BHA), glutathione, ubiquinone, carotenoids and vitamin E as well as functional equivalents and/or derivatives thereof. By “radical formation” is meant the process by which a free radical is formed by oxidation and/or oxidative processe(s).

Accordingly, the pharmaceutical compositions of the invention are less prone to oxydative degradation than the counterpart composition lacking the radical quencher according to the invention. Physically and chemically more stable formulations have a considerably longer shelf life, thereby reducing their production cost and making these formulations more affordable in a world where healthcare expenditures are capped. In addition to the economic advantages, the superior formulations of the invention remain stable at the range of temperatures to which these type of medications are exposed on a daily basis by the average patient. Medicaments are generally kept with the patient during the day and are often exposed to extreme low temperatures (e.g., a cold Boston day) or extreme high temperatures and humidity (e.g., a July day in Miami).

One of ordinary skill in the art can easily determine the presence of oxydative degradation products by HPLC analysis. For example, the glucocorticosteroid budesonide, 21-dehydrobudesonide is a readily recognizable degradation product of budesonide (see drug master file for budesonide available from the raw material manufacturer Industriale Chimica d.r.l. & Sicor S.p.A.).

In preferred embodiments of the first, second, and third aspects of the invention, the radical quencher is ascorbyl palmitate or Vitamin E acetate.

In accordance with the invention, any fluoroalkane propellant that is suitable for inhalation can be used. Examples of suitable fluoroalkanes (hydrofluoroalkanes) include, without limitation, 1,1,1,2 tetrafluoroethane (“HFA-134a”), 1,1,1,2,3,3,3 heptafluoropropane (“HFA-227ea”), pentafluoroethane (“HFA-125”), 1,1-difluoroethane (“HFA-152a”), and difluoromethane (“HFA-32”). Hydrocarbon and/or aliphatic gases may be added to modify propellant characteristics as required. Preferably, the aerosol formulation is substantially free of chlorofluorocarbons, which, unlike hydrofluoroalkanes, have been implicated in the depletion of the ozone layer. (For a general discussion, see the Montreal Protocol on Substances that Deplete the Ozone Layer published by the Liaison Office of the United Nations Environmental Program, New York, N.Y. (1989)). However, if desired, chlorofluorocarbons can be utilized. Preferably, the fluoroalkane is 1,1,1,2-tetrafluoroethane (HFA-134a) or 1,1,1,2,3,3,3-heptafluoropropane (HFA-227ea). It is understood that the nature of the propellant used is not an essential element of the invention. Hence, although at the present time HFA's are preferred propellants, other can be used in their place without affecting the basic formulation approach described herein.

In various embodiments of the first, second, and third aspects of the invention, the propellant is 1,1,1,2-tetrafluoroethane, 1,1,1,2,3,3,3-heptafluoro-n-propane or a mixture thereof.

The propellant is usually present in an amount of from about 60% to about 97% by weight, preferably from about 70 to about 90% by weight, based on the total weight of the aerosol formulation.

In accordance with the invention, any cosolvent that is suitable for inhalation and capable of dissolving or solubilizing the glucocorticosteroid in the mixture of cosolvent and propellant can be used. Examples of suitable cosolvents include alcohols, ethers, hydrocarbons, and perfluorocarbons. Preferably, the cosolvent is a short chain polar alcohol. More preferably, the cosolvent is an aliphatic alcohol having from one to six carbon atoms, such as ethanol or isopropanol. The most preferred cosolvent is ethanol. Examples of suitable hydrocarbons include n-butane, isobutane, pentane, neopentane and isopentanes. Examples of suitable ethers include dimethyl ether and diethyl ether. Examples of suitable perfluorocarbons include perfluoropropane, perfluorobutane, perfluorocyclobutane, and perfluoropentane.

In various embodiments of the first, second, and third aspects of the invention, the cosolvent is an alcohol. In certain embodiments, the alcohol is a C₂-C₆ alcohol. In certain embodiments, the alcohol is, without limitation, ethanol, isopropanol, or propylene glycol.

In one non-limiting example, ethanol is utilized as a cosolvent. When ethanol is utilized as the cosolvent, the cosolvent is usually present in an amount of from about 0.01% to about 40%, preferably from about 3% to about 25% by weight, based on the total weight of the aerosol formulation. The cosolvent (e.g., ethanol) should be present in an amount which fully dissolves or solubilizes the glucocorticosteroid in the mixture of ethanol and propellant. Preferably, cosolvent is present in amount sufficient to fully maintain the glucocorticosteroid in solution at freezing temperatures, such as 0° C. In general, as the temperature is decreased, the solubility of glucocorticosteroid in the cosolvent is decreased. Therefore, an excess of cosolvent (e.g., ethanol) over the amount required to fully dissolve or solubilize the glucocorticosteroid at ambient or room temperature is preferred. In this regard, where the cosolvent is ethanol, the cosolvent is preferably present in an amount of at least 10% by weight, more preferably at least 15% by weight, even more preferably at least 20% by weight, and most preferably at least 25% by weight. Based on the disclosure provided herein, one skilled in the art will recognize that lower concentrations of medicament usually require lower concentrations of cosolvent, and vice versa, in order to form a stable solution. Furthermore, one skilled in the art will recognize that the type of propellant utilized can also affect the amount of cosolvent required to fully dissolve or solubilize the glucocorticosteroid in the mixture of cosolvent and propellant.

In general, the greater the polarity of the propellant the less cosolvent required to fully dissolve or solubilize the glucocorticosteroid. For example, when HFA-134a is utilized as the propellant and ethanol is utilized as the cosolvent in a formulation of the invention, the amount of ethanol is preferably from about 10 to about 30% by weight. When HFA-227ea is utilized as the propellant and ethanol is utilized as the cosolvent in a formulation of the invention, the amount of ethanol is preferably from about 6 to about 20% by weight.

A preferred formulation of the invention comprises as a propellant, either HFA-134a or HFA-227ea in an amount less than about 90% by weight; as a cosolvent, ethanol in an amount of at least about 10% by weight; as a glucocorticosteroid, budesonide in an amount of from about 0.05 to about 0.5% by weight; and as a radical quencher, either vitamin E-acetate or ascorbyl palmitate in an amount of from about 0.01 to about 1% by weight. A particularly preferred formulation comprises about 86% by weight of HFA-227ea, about 14% by weight of ethanol, and about 0.3% by weight of budesonide. Another particularly preferred formulation comprises about 75% by weight of HFA-134a, about 25% by weight of ethanol, and about 0.3% by weight of budesonide.

Pressurized metered dose inhalers are now well known in the art and are useful for administering a formulation of the invention, where the formulation of the invention is an aerosol formulation. Any pressurized metered dose inhaler that is suitable for application of medicaments to the lungs or nose of a patient can be used. Pressurized metered dose inhalers usually are equipped with an actuator having a spray orifice diameter of about 460 μm. However, with the higher concentrations of solvent employed in the present invention, it may be desirable that the solvent evaporates as soon as possible after inhalation. This can be achieved by reducing particle size by reducing the spray orifice diameter, for example, to 250 μm, in combination with using solvent concentrations greater than about 10% by weight.

Based on the disclosure provided herein, one skilled in the art will be able to adjust the component composition to deliver a desired dose for the selected metered valve, without undue experimentation. For example, the composition may be altered to adjust the vapor pressure of the formulation. The aerosol formulation and metering valve are usually selected to provide a therapeutically effective amount of the budesonide per actuation. An example of a therapeutically effective amount of budesonide is about 50 to about 400 μg per activation, preferably about 100 to about 250 μg per activation.

The pressurized metered dose inhaler can be formed by any suitable method. For example, the selected amount of budesonide can be weighed and inserted into a suitable container, such as a glass bottle or aluminum canister. The use of containers coated with a polymer (such as TEFLON®) has been found to confer a limited additional protection of the formulation as evidenced by the reduction of the oxydative degradation products observed. The cosolvent can then be weighed and added to the container. Once all of the non-gaseous components have been added to the container, the metered valve can be crimped on to seal the container. Then, the desired amount of propellant can be added to the container through the metered valve. The budesonide can be dissolved or solubilized into the mixture of cosolvent and propellant by agitating the formulation, such as by sonication. For the small scale preparations, about 5 minutes of sonication has been found to be suitable to dissolve or solubilize a formulation having a total weight of about 12 grams. Alternative well known methods of homogenizing the formulation of the invention may be substituted on a commercial scale production.

The elements in the second and third aspects on the present inventions are essentially as set forth in the first aspect of the invention. In a first aspect, the invention provides a pharmaceutical composition which remains chemically and physically stable under standard conditions comprising any glucocorticosteroid (preferably budesonide), any propellant (preferably, fluoroalkane propellant-1,1,1,2-tetrafluoroethane, 1,1,1,2,3,3,3-heptafluoro-n-propane or a mixture thereon, any cosolvent (preferably alcohols, ethers, hydrocarbons, and perfluorocarbons) and any radical quencher (preferably ascorbyl palmitate or Vitamin E acetate).

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the following examples and claims.

### EXAMPLE I

### Degradation-Resistance of a Glucocorticosteroid in a Formulation of the Invention

Laboratory formulations according to the present invention were generated by combining the components shown in Tables I and V, utilizing a series of steps. The initial step involves separately adding each component of the formulation within a bottle or canister. The first steps include: adding weighed the glucocorticosteroid into a glass bottle or an aluminum canister which may or may not be coated with a plastic coating, adding the weighed radical quencher to the bottle or canister, and adding the weighed cosolvent to the bottle or canister. The following step involves crimping a valve upon the bottle or canister. Next, the step of sonicating the formulating for approximately two minutes. Finally, the step of adding a known amount of propellant through the valve into the bottle or canister and sonicating the formulation for approximately two minutes. Note that the balance is preferably tared with the canister between subsequent additions of an ingredient.

Ascorbyl Palmitate as the Radical Quencher

Using ascorbyl palmitate as the radical quencher, budesonide as the glucocorticosteroid, ethanol as the cosolvent, and HFA-134a as the propellant, two formulations (A and B) were made in canisters that were either coated or uncoated with a polymer on the interior wall. The components of the Formulations A and B are shown in Table I.

TABLE I
Formulations A and B

| | Formulation A | Formulation B |
|---|---|---|
| | (approximate | (approximate |
| Component | % by weight) | % by weight) |
| HFA-134a | 78.7 | 74 |
| Ethanol | 21 | 25 |
| Ascorbyl Palmitate | 0.0 | 0.6 |
| Budesonide | 0.3 | 0.3 |

Formulations A and B were subjected to stability study conditions of 40° C. in a laboratory oven. At various time points, formulation samples were tested for average shot weight (mg), dose delivered/actuation (μg/actuation), percentage of total epimers of budesonide (R and S), and percentage material balance based on shot weights. The material balance indicates the total percentage of drug recovered from the valve stem, actuator, and dose tube used in Dose Uniformity Testing.

The results of these stability studies for 7, 28, 63, 92 and 180 days on Formulation A are shown in Table II (in the uncoated canister) and Table III (coated canister). Table IV shows a comparison of the total impurities (identified and unknown) after 7, 28, 63, 92 and 180 days of stability study conditions on Formulation A from uncoated and coated canisters.

TABLE II
Stability of Formulation A in Uncoated Container

| Days in Stability | 7 | 28 | 63 | 92 | 180 |
|---|---|---|---|---|---|
| Average Shot | 67.53 | 65.87 | 67.42 | 70.19 | 71.91 |
| Weight (mg) | | | | | |
| Dose | 172.11 | 166.23 | 172.15 | 155.90 | 130.65 |
| delivered/actuation | | | | | |
| (μg/actuation) | | | | | |
| % R-Epimer | 43.05 | 43.75 | 44.82 | 45.59 | 47.05 |
| Average Material | 106.97 | 105.96 | 107.18 | 93.21 | 76.27 |
| Balance based on | | | | | |
| Shot Weight (%) | | | | | |

TABLE III
Stability of Formulation A in Coated Canisters

| Days in Stability | 7 | 28 | 63 | 92 | 180 |
|---|---|---|---|---|---|
| Average Shot | 68.58 | 61.71 | 68.40 | 60.35 | 70.38 |
| Weight (mg) | | | | | |
| Dose | 193.07 | 169.73 | 185.31 | 160.15 | 168.18 |
| delivered/actuation | | | | | |
| (μg/actuation) | | | | | |
| % R-Epimer | 43.13 | 43.34 | 43.31 | 43.63 | 44.23 |
| Average Material | 110.27 | 107.96 | 106.11 | 104.63 | 94.81 |
| Balance based on | | | | | |
| Shot Weight (%) | | | | | |

TABLE IV
Degradation Profile of Formulation A in Coated
and Uncoated Canisters

| | | 21-Dehydro- | Unknown | |
|---|---|---|---|---|
| | | budesonide | Impurity | |
| | Days in | (total of 2 | (total of 2 | Total |
| Lot Number | Stability | epimers) (%) | epimers) (%) | Impurities |
| Formulation | 7 | 0.00 | 0.00 | 0.00 |
| A in Uncoated | 28 | 2.00 | 0.17 | 2.17 |
| Canister | 63 | 5.79 | 2.08 | 7.87 |
| | 92 | 8.50 | 2.90 | 11.40 |
| | 180 | 9.95 | 4.25 | 14.20 |
| Formulation | 7 | 0.00 | 0.00 | 0.00 |
| A in Coated | 28 | 0.00 | 0.00 | 0.00 |
| Canister | 63 | 1.12 | 0.57 | 1.69 |
| | 92 | 1.40 | 0.40 | 1.80 |
| | 180 | 2.68 | 2.20 | 4.88 |

The results of these stability studies for 7, 28, 63, 92 and 180 days on Formulation B (prepared in uncoated and coated aluminum canisters) are shown in Tables V and VI, respectively. Table VII shows a comparison of the total impurities (identified and unknown) after 7, 28, 63, 92 and 180 days of stability study conditions on Formulation B from uncoated and coated canisters.

TABLE V
Stability of Formulation B Uncoated

| Days in Stability | 7 | 28 | 63 | 92 | 180 |
|---|---|---|---|---|---|
| Average Shot | 66.62 | 63.65 | 61.05 | 60.60 | 69.42 |
| Weight (mg) | | | | | |
| Dose | 221.13 | 187.51 | 177.86 | 178.06 | 161.06 |
| delivered/actuation | | | | | |
| (μg/actuation) | | | | | |
| % R-Epimer | 43.10 | 43.19 | 43.40 | 43.40 | 43.43 |
| Average Material | 114.53 | 101.57 | 99.86 | 101.31 | 80.02 |
| Balance based on | | | | | |
| Shot Weight (%) | | | | | |

TABLE VI
Stability of Formulation B Coated

| Days in Stability | 7 | 28 | 63 | 92 | 180 |
|---|---|---|---|---|---|
| Average Shot | 67.09 | 61.78 | 66.81 | 67.60 | 68.99 |
| Weight (mg) | | | | | |
| Dose | 202.39 | 175.36 | 190.36 | 183.13 | 197.61 |
| delivered/actuation | | | | | |
| (μg/actuation) | | | | | |
| % R-Epimer | 43.20 | 43.23 | 43.17 | 43.20 | 43.37 |
| Average Material | 106.27 | 99.55 | 100.41 | 95.44 | 100.89 |
| Balance based on | | | | | |
| Shot Weight (%) | | | | | |

TABLE VII
Degradation Profile of Formulation B in Coated
and Uncoated Canister

| | | 21-Dehydro- | Unknown | |
|---|---|---|---|---|
| | | budesonide | Impurity | |
| | Days in | (total of 2 | (total of 2 | Total |
| Lot Number | Stability | epimers) (%) | epimers) (%) | Impurities |
| 579-43B | 7 | 0.00 | 0.00 | 0.00 |
| (Uncoated) | 28 | 0.00 | 0.00 | 0.00 |
| | 63 | 0.31 | 0.00 | 0.31 |
| | 92 | 0.31 | 0.00 | 0.31 |
| | 180 | 0.22 | 0.39 | 0.61 |
| 579-43B | 7 | 0.00 | 0.00 | 0.00 |
| (Coated) | 28 | 0.00 | 0.00 | 0.00 |
| | 63 | 0.00 | 0.00 | 0.00 |
| | 92 | 0.00 | 0.00 | 0.00 |
| | 180 | 0.25 | 0.75 | 1.00 |

Studies after 14 days of exposure to 40° C. did not exhibit degradation products in both the control formulation (Formulation A) and the formulation containing the radial quencher (Formulation B). Moreover, after 28 days of treatment at 40° C., Formulation B exhibited an average material balance of 100.56% (average of 101.57% in uncoated canister and 99.55% in coated canister). Control Formulation A containing no radial quencher showed an average material balance of 106.96% after 28 days average of 105.96% in uncoated canister and 107.96% in coated canister).

Surprisingly, no degradation products were observed in Formulation B after 28 days of oven treatment at 40° C. compared with 2.17% total degradation products (both 21-Dehydrobudesonide and an unknown impurity) observed in the control formulation (Formulation A).

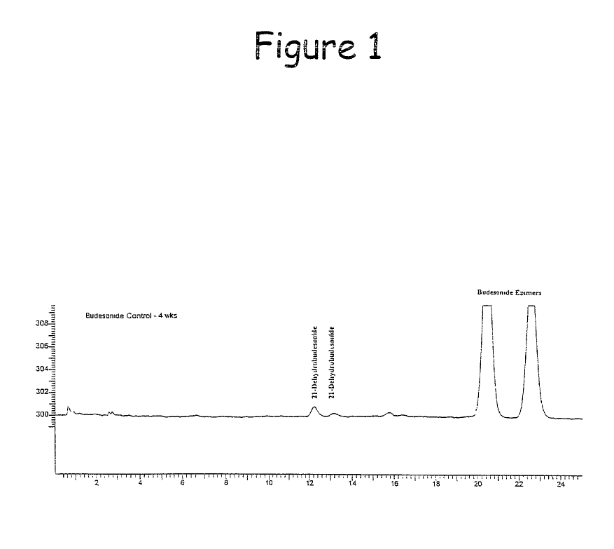

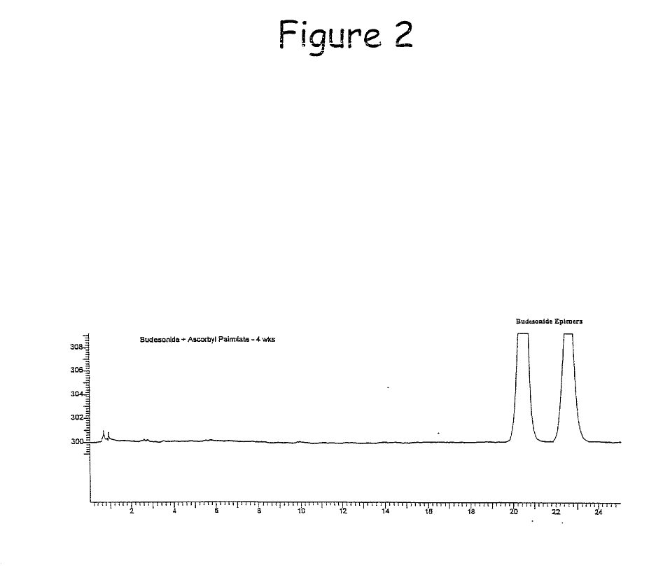

This difference in the extent of degradation observed for Formulation A and Formulation B after 28 days at 40° C. is illustrated in FIGS. 1 and 2 respectively. In the HPLC chromatogram of FIG. 1, the pure, non-degraded product eluted as two completely resolved peaks of the isomers (R and S) at approximately 19-23 minutes after injection. The two peaks corresponding to the R and the S isomer of the degradation product, 21-Dehydrobudesonide, appeared at approximately 12-14 minutes after injection. In the HPLC chromatogram of FIG. 2, the pure, non-degraded product eluted as two completely resolved peaks of the isomers (R and S) at approximately 19-23 minutes after injection. Notably absent are the two peaks corresponding to the R and the S isomer of the degradation product, 21-Dehydrobudesonide normally eluting at approximately 12-14 minutes after injection.

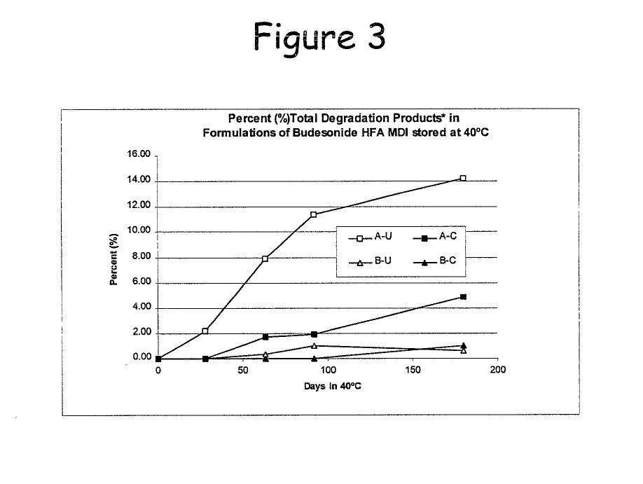

Moreover, even after 180 days of 40° C. oven treatment, the total percent degradation exhibited by Formulation B, containing the radical quencher, was only 0.61 and 1.00 in both uncoated and coated canisters, respectively. Comparatively, the total percent degradation observed from Formulation A, containing no radical quencher, was 14.20 and 4.88 in both uncoated and coated canisters, respectively. This remarkable difference in percent degradation observed is graphically shown in FIG. 3 (see graph below). FIG. 3 shows the percent total degradation of the various formulations on coated versus uncoated canisters (AU=Formulation A in an uncoated canister; BU=Formulation B in an uncoated canister; AC=Formulation A in a coated canister; BC=Formulation B in a coated canister.)

Other Formulations

Based on successful preformulation studies using ascorbyl palmitate and Vitamin E-acetate (data not shown), other formulations containing these radical quenchers were also prepared with HFA-134a, HFA-227ea, or a combination of both propellants, as described in Table VIII. Representative glucocorticosteroidical formulations containing HFA and either ascorbyl paltiate or Vitamin E—acetate are tabulated hereafter.

TABLE VIII
Representative Stabilized Formulations Containing HFA Propelent(s)

| | Ingredient | Approximate % by Weight |
|---|---|---|
| Formulation 1 | HFA-Propellant* | 60-90 |
| | Ethanol | 10-40 |
| | Ascorbyl Palmitate | 0.6 |
| | Glucocorticosteroid | 0.3 |
| Formulation 2 | HFA-Propellant* | 60-90 |
| | Ethanol | 10-40 |
| | Vitamin E-acetate | 0.6 |
| | Glucocorticosteroid | 0.3 |

*HFA-Propellant included HFA-134a, HFA-227ea, or a combination of both.

Analytical studies of these formulations as compared to control formulations (all ingredients are identical except for the absence of the radical quencher) following a stress treatment (14 days at 40° C.) showed a significant reduction of degradation in the presence of a radical quencher (data not shown).

Similar results were obtained when using USP intentional degradation methodologies (such as acid, base, peroxide) (data not shown).

### EXAMPLE II

Use of Representative Formulations for Aerosol Delivery

To illustrate the use of radical quencher-containing formulations according to the invention, representative formulation B is administered to patients in need of budesonide regimens. One of skill in the art will appreciate that while this example is written for the administration of a budesonide-containing formulation, similar formulations containing other glucocorticosteroids—as discussed in more detail above-may be easily formulated and administered as described herein and according to standard methodologies known in the field. Similarly, while this example is written for a formulation of a 100 or 200-μg/ actuation budesonide-HFA solution containing a radical quencher, different concentrations, delivery dosages and forms may be easily tailored to meet a specific medical condition or a particular patient's requirement(s).

Patients suffering from a bronchial disorder such as asthma are orally administered Formulation B (see above) two puffs twice a day (200-μg/ actuation or 100-μg/ actuation depending on the severity of the patient's conditions). Improvement in asthma control following inhaled administration of Formulation B is expected to occur within 24 hours of beginning treatment although, maximum benefit may not be achieved for one to two weeks or longer after starting treatment. After asthma stability has been achieved the starting dose it is always desirable to titrate to the lowest effective dose to reduce the possibility of side effects.

While the claimed invention has been described in detail and with reference to specific embodiments thereof, it will be apparent to one of ordinary skill in the art that various changes and modifications can be made to the claimed invention without departing from the spirit and scope thereof.

What is claimed is:

1. A pharmaceutical composition comprising a glucocorticosteroid, a propellant, a cosolvent, and a radical quencher.

2. The pharmaceutical composition of claim 1, wherein the radical quencher is ascorbyl palmitate.

3. The pharmaceutical composition of claim 1, wherein the radical quencher is Vitamin E.

4. The pharmaceutical composition of claim 1, wherein the radical quencher is selected from the group consisting of ascorbic acid, ascorbyl palmitate, sodium bisulfite, butylated hydroxytoluene (BHT), bytylated hydroxyanisole (BHA), glutathione, ubiquinone, carotenoids and vitamin E and functional equivalents and/or derivatives thereof.

5. The pharmaceutical composition of claim 1, wherein the glucocorticosteroid is selected from the group consisting of budesonide, testosterone, progesterone, estrogen, flunisolide, triamcinolone, beclomethasone, betamethasone, dexamethasone, fluticasone, methylprednisolone, prednisone, hydrocortisone, ciclesonide, mometasone, desonide, and rofleponide.

6. The pharmaceutical composition of claim 1, wherein the glucocorticosteroid is budesonide.

7. The pharmaceutical composition of claim 1, wherein the propellant is 1,1,1,2-tetrafluoroethane, 1,1,1,2,3,3,3-heptafluoro-n-propane or a mixture thereof.

8. The pharmaceutical composition of claim 1, wherein the cosolvent is a polyol.

9. The pharmaceutical composition of claim 7, wherein the polyol is a C₂-C₆ alcohol.

10. The pharmaceutical composition of claim 7, wherein the polyol is selected from the group consisting of ethanol, isopropanol, and propylene glycol.

11. A pressurized metered dose inhaler comprising a container equipped with a metering valve and containing a pressurized aerosol formulation comprising a glucocorticosteroid, a propellant, a cosolvent, and a radical quencher.

12. The pressurized metered dose inhaler according to claim 11, wherein the glucocorticosteroid is selected from the group consisting of budesonide, testosterone, progesterone, estrogen, flunisolide, triamcinolone, beclomethasone, betamethasone, dexamethasone, fluticasone, methylprednisolone, prednisone, hydrocortisone, ciclesonide, mometasone, desonide, and rofleponide.

13. The pressurized metered dose inhaler according to claim 11, wherein the glucocorticosteroid is budesonide.

14. The pressurized metered dose inhaler according to claim 11, wherein the propellant is 1,1,1,2-tetrafluoroethane, 1,1,1,2,3, 3,3-heptafluoro-n-propane or a mixture thereof.

15. The pressurized metered dose inhaler according to claim 11, wherein the cosolvent is a polyol.

16. The pressurized metered dose inhaler according to claim 15, wherein the polyol is a C₂-C₆ alcohol.

17. The pressurized metered dose inhaler according to claim 15, wherein the polyol is selected from the group consisting of ethanol, isopropanol, and propylene glycol.

18. The pressurized metered dose inhaler according to claim 11, wherein the radical quencher is ascorbyl palmitate.

19. The pressurized metered dose inhaler according to claim 18, wherein the radical quencher is Vitamin E acetate.

20. A method for the treatment of a bronchial disorder or an inflammatory bowel disorder in a mammal by administering a pharmaceutical formulation comprising a glucocorticosteroid, a propellant, a cosolvent, and a radical quencher.

21. The method according to claim 20, wherein the glucocorticosteroid is selected from the group consisting of budesonide, testosterone, progesterone, estrogen, flunisolide, triamcinolone, beclomethasone, betamethasone, dexamethasone, fluticasone, methylprednisolone, prednisone, hydrocortisone, ciclesonide, mometasone, desonide, and rofleponide.

22. The method according to claim 20, wherein the glucocorticosteroid is budesonide.

23. The method according to claim 20, wherein the propellant is 1,1,1,2-tetrafluoroethane, 1,1,1,2,3,3,3-heptafluoro-n-propane or a mixture thereof.

24. The method according to claim 20, wherein the cosolvent is a polyol.

25. The method according to claim 24, wherein the polyol is a C₂-C₆ alcohol.

26. The method according to claim 24, wherein the polyol is selected from the group consisting of ethanol, isopropanol, and propylene glycol.

27. The method according to claim 20, wherein the radical quencher is ascorbyl palmitate.

28. The method according to claim 20, wherein the radical quencher is Vitamin E acetate.
